(19) United States
(12) Patent Application Publication
(10) Pub. No.: US 2024/0374355 A1
(43) Pub. Date: Nov. 14, 2024

(54) SCREW FOR DENTAL IMPLANT

(71) Applicant: Cheon-Ho MOON, Hanam-si Gyeonggi-do (KR)

(72) Inventor: Cheon-Ho MOON, Hanam-si Gyeonggi-do (KR)

(21) Appl. No.: 18/688,237

(22) PCT Filed: Dec. 7, 2022
(86) PCT No.: PCT/KR2022/019758

§ 371 Date: Feb. 29, 2024

(30) Foreign Application Priority Data
Dec. 20, 2021 (KR) 10-2021-0182321

(51) Int. Cl. A61C 8/00

(52) U.S. Cl. CPC A61C 8/0028

(57) ABSTRACT

The present invention relates to a screw for a dental implant, comprising: a body (122) in which a through hole (124) is formed in the vertical direction; and a cover part (129) formed at an edge on a top end of the body (122), wherein the cover part (129) comprises: a protective body (120) formed by including a horizontal plate (127) formed to have a predetermined area in the horizontal direction and an inclined plate (128) formed downward at an end of the horizontal plate (127); and a connecting bolt (140) that fixes the protective body (120) to a fixture (200) by passing through the through hole (124) to be fastened to the fixture (200) implanted in the alveolar bone, and thus, when the protective body (120) is fixed to the fixture (200) by means of the connecting bolt (140) in the process of implant placement surgery, the cover part (129) covers and protects a bone graft material (320) implanted around the fixture (200), thereby assisting in regeneration of the alveolar bone.

### TECHNICAL FIELD

The present disclosure relates to a screw for a dental implant and, more particularly, to a screw for a dental implant that induces high-quality alveolar bone regeneration by covering and protecting a bone graft material filled in a unilateral alveolar bone defect part during dental implant placement surgery.

### BACKGROUND ART

A dental implant functions by being fixed to the jawbone through osseointegration with the alveolar bone, and for the success of implant treatment, a sufficient amount of the alveolar bone is required to exist in an area in which an implant is to be placed. However, in most cases, as the amount of the alveolar bone to accommodate the implant is insufficient, bone expansion or bone augmentation surgery is required for implant treatment.

In the bone expansion or bone augmentation surgery described above, after bone grafting is performed on an alveolar bone defect part, a bone graft material is protected with an absorbable barrier, non-absorbable barrier, or titanium mesh to prevent surrounding soft tissue from moving to the bone graft part and to help bone formation.

Depending on a product, an absorbable barrier is usually gradually absorbed between 2 and 24 weeks, so additional removal surgery is not necessary. However, when the absorbable barrier is absorbed too quickly or has a problem with soft tissue sealing, the absorbable barrier may be exposed to the outside at an early stage, which may interfere with alveolar bone formation. In the case of a non-absorbable barrier, additional removal surgery is required later since the non-absorbable barrier is not naturally absorbed. Meanwhile, most absorbent or non-absorbable barriers have the disadvantage that they are expensive to varying degrees.

When using titanium mesh, an operator recognizes the shape of a location at which an alveolar bone defect occurs, trims the location to a desired shape, and then bends the location again in three dimensions to cover the defect part with a bone graft material applied to the defect part. Next, separate screws are used to finally secure the titanium mesh. However, when titanium mesh is used, it is difficult to completely cover the alveolar bone defect. In addition, surgery time increases, and the bending area may locally protrude excessively. This phenomenon is a major cause of alveolar bone regeneration failure due to the protrusion part breaking through the gums (gingiva) and being infected by the outside during the period of alveolar bone regeneration induction. In addition, in order to remove the screws fastened to secure the titanium mesh, a wider gum incision is required to be made during a second surgery. As a result, not only does surgery time increase, but edema due to bleeding occurs and causes pain to the patient, putting a burden on both an operator and a patient.

Meanwhile, Korean Patent No. 10-1280620 discloses a temporary fastening member that is temporarily fastened to a fixture during the osseointegration period of the fixture implanted in the alveolar bone during dental implant surgery. The temporary fastening member has a structure that covers the entire upper part of the fixture and is intended to protect a bone graft material from the external force of a vertical upper side. However, the temporary fastening member has a problem in that it cannot completely block pressure or stimulation from the side of the bone graft material, and has limitations in widespread application since the temporary fastening member cannot cover only one side when there is sufficient alveolar bone on one side around the fixture and insufficient alveolar bone on the other side around the fixture.

### DISCLOSURE

### Technical Problem

The present disclosure is intended to provide a means to stably protect a bone graft material grafted to an alveolar bone defect part in a case in which the alveolar bone defect part exists on one side of a fixture placement point due to insufficient width and inclination of the remaining alveolar bone at an implant surgery site.

### Technical Solution

In order to accomplish the above objective, the present disclosure provides a screw for a dental implant, the screw including: a body in which a through hole is formed in a vertical direction, and a cover part formed on an edge of a top of the body, wherein the cover part includes a protective body formed by comprising a horizontal plate formed to have a predetermined area in a horizontal direction and an inclined plate formed downward on an end of the horizontal plate, and a connecting bolt that fixes the protective body to the fixture by passing through the through hole to be fastened to the fixture implanted in the alveolar bone, and when the protective body is fixed to the fixture by the connecting bolt in a process of implant placement surgery, the cover part covers and protects a bone graft material implanted around the fixture.

### Advantageous Effects

The screw for a dental implant according to the present disclosure can safely protect the bone graft material implanted around the fixture when it is necessary to induce alveolar bone regeneration unilaterally on an implant surgery site, and furthermore, block stimulation or pressure applied to the bone graft material from a vertically upper side and a lateral side, thereby enabling the regeneration of high-quality alveolar bone.

### DESCRIPTION OF DRAWINGS

FIG. 1 is an example view showing a dissembled state of a screw for a dental implant according to the present disclosure,

FIG. 2 is an example view showing cross-sectional configuration of a protective body constituting the screw for a dental implant according to the present disclosure,

FIG. 3 is a cross-sectional view of the combined state of the screw for a dental implant according to the present disclosure,

FIGS. 4 and 5 are example views showing the use of the screw for a dental implant according to the present disclosure,

FIG. 6 is an example view of a connecting bolt according to another embodiment of the present disclosure, and

FIG. 7 is an example view of the combined state of the screw for a dental implant to which the connecting bolt is applied according to the another embodiment of the present disclosure.

### MODE FOR INVENTION

The present disclosure provides a screw for a dental implant, the screw including: a body in which a through hole is formed in a vertical direction, and a cover part formed on an edge of a top of the body, wherein the cover part includes a protective body formed by including a horizontal plate formed to have a predetermined area in a horizontal direction and an inclined plate formed downward on an end of the horizontal plate, and a connecting bolt that fixes the protective body to the fixture by passing through the through hole to be fastened to the fixture implanted in the alveolar bone, and when the protective body is fixed to the fixture by the connecting bolt in a process of implant placement surgery, the cover part covers and protects a bone graft material implanted around the fixture.

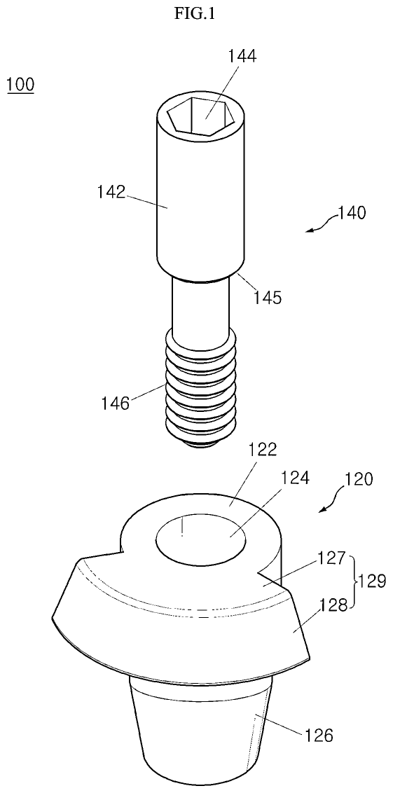

Hereinafter, the present disclosure will be described in detail with reference to FIGS. 1 to 7.

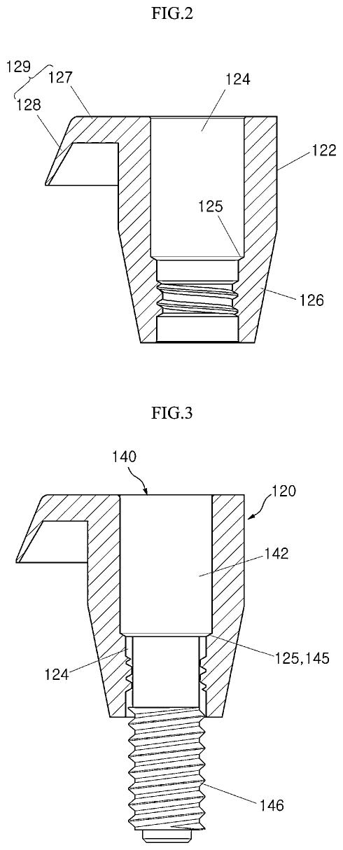

FIG. 1 is an example view showing a dissembled state of the screw for a dental implant according to the present disclosure, FIG. 2 is an example view showing cross-sectional configuration of a protective body constituting the screw for a dental implant according to the present disclosure, and FIG. 3 is a cross-sectional view of the combined state of the screw for a dental implant according to the present disclosure.

As shown, the screw 100 for a dental implant according to the present disclosure includes the protective body 120 and the connecting bolt 140. The protective body 120 and the connecting bolt 140 are preferably made of metal materials, but are not limited thereto and may be made of bio-friendly materials.

The protective body 120 has the body 122 formed in a three-dimensional shape. The body 122 is formed to have a predetermined length in a vertical direction, and may be circular or polygonal in a cross-sectional shape. In addition, a fixture connection part 126 configured to have thickness decreasing gradually downward is formed on the lower part of the body.

The body 122 has through hole 124 formed by passing through the body 122 in a vertical direction. The through hole 124 has large diameter at an upper part thereof and has a small diameter at a lower part thereof to have a jaw 125 formed therebetween, and threads may be formed on the lower part of the through hole.

The cover part 129 is formed on the upper end of the body 122. The cover part 129 is formed at an edge of the body 122 by protruding in a horizontal direction therefrom, and includes the horizontal plate 127 formed to have a predetermined area in the horizontal direction and the inclined plate 128 formed downward from an end of the horizontal plate 127. The horizontal plate 127 may be formed into a fan shape, and in this case, a central angle thereof is preferably formed in the range of 10 to 180 degrees. In addition, the inclined plate 128 may be formed to be inclined in a direction away from the body 122 downward.

The connecting bolt 140 has a head 142 formed at an upper end thereof and male threads 146 formed at a lower part thereof. The head 142 is formed to be thicker than other parts and has a jaw 145 formed at a lower end thereof. A wrench groove 144 is formed in the head 142 so that the head 142 can be rotated with a tool such as a hex wrench.

The connecting bolt 140 is coupled to the body 122 by passing through the through hole 124 formed in the body 122. In this case, the male threads 146 formed at the lower part passes through the through hole 124 and is exposed to the outside, and the head 142 is completely accommodated and combined within the through hole 124. In addition, the jaw 145 formed on the connecting bolt 140 is held by the jaw 125 formed on the through hole 124, so that the connecting bolt 140 is coupled thereto without falling below the through hole 124.

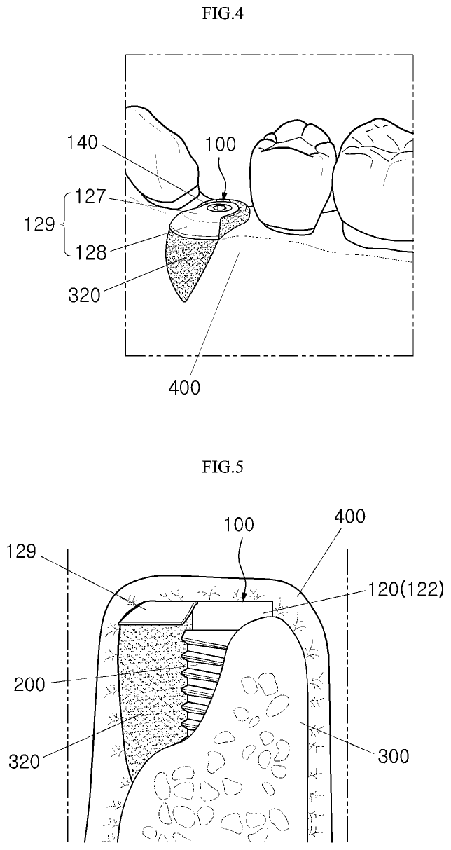

FIGS. 4 and 5 are example views showing the use of the screw for a dental implant according to the present disclosure.

In the process of implant surgery, the fixture 200 is embedded in the alveolar bone 300 and fixed thereto. In this state, the screw 100 for a dental implant according to the present disclosure is used.

During implant surgery, the fixture 200 is embedded in the alveolar bone 300. The gum 400 is incised and the fixture 200 is embedded in the alveolar bone 300.

The fixture 200 has a screw structure, and a hole having a predetermined depth in a vertical direction is formed in a body thereof. In addition, female threads are formed in the hole.

The connecting bolt 140 passes through the through hole 124 formed in the protective body 120 and is engaged with the female threads formed on the fixture 200. The male threads 146 formed on the lower part of the connecting bolt 140 is coupled to and engaged with the female threads. Accordingly, the body 122 is embedded into the hole formed in the fixture 200 and is fixed to the fixture 200. In this case, the body 122 may be coupled to the fixture 200 while the fixture connection part 126 is completely accommodated in the hole formed in the fixture 200 and the upper part of the body 122 is exposed to the outside.

When the screw 100 for a dental implant is coupled to the fixture 200 as described above, the horizontal plate 127 formed on the protective body 120 covers the top of the bone graft material 320 implanted around the fixture 200. At the same time, the inclined plate 128 covers a side of the bone graft material 320. As a result, the cover part 129 covers and protects the top and side of the bone graft material 320 to block external pressure and stimulation. This helps in regenerating the alveolar bone.

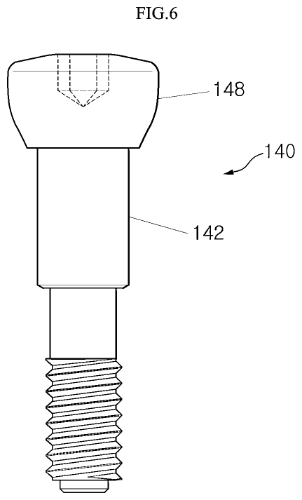

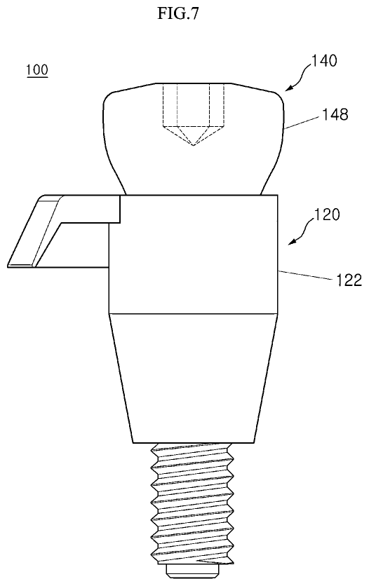

FIG. 6 is an example view of a connecting bolt according to another embodiment of the present disclosure, and FIG. 7 is an example view of the combined state of the screw for a dental implant to which the connecting bolt is applied according to the another embodiment of the present disclosure.

The screw 100 for a dental implant according to the present disclosure may further include a sealing head 148 formed on the top of the connecting bolt 140. When the connecting bolt 140 has the head 142 formed therein, the sealing head 148 may be formed on the head 142, and in this case, the sealing head 148 is formed to be larger than the head 142. The sealing head 148 is formed in a shape that matches the shape of a part pierced in the gum.

When the connecting bolt 140 is coupled to the body 122 of the protective body 120, the sealing head 148 protrudes from the top of the body 122, and when the screw 100 for a dental implant according to the present disclosure is coupled to the fixture 200 during implant placement surgery simultaneously with tooth extraction, the sealing head 148 formed as described above is embedded into the gum that is pierced, that is, an opening formed in the gum, and functions to seal the opening.

<Description of the Reference Numerals in the Drawings>

| | |
|---|---|
| 100: Screw for a dental implant, | |
| 120: Protective body, | 122: Body, |
| 124: Through hole, | 125: Jaw, |
| 126: Fixture connection part, | 127: Horizontal plate, |
| 128: Inclined plate, | 129: Cover part, |
| 140: Connecting bolt, | 142: Head, |
| 144; Wrench groove, | 145: Jaw, |
| 146: Male thread, | 148: Sealing head, |
| 200: Fixture, | 300: Alveolar bone, |
| 320: Bone graft material, | 400: Gum. |

1. A screw for a dental implant, the screw comprising:
a body (122) in which a through hole (124) is formed in a vertical direction, and
a cover part (129) formed on an edge of a top of the body (122),
wherein the cover part (129) comprises a protective body (120) formed by comprising a horizontal plate (127) formed to have a predetermined area in a horizontal direction and an inclined plate (128) formed downward on an end of the horizontal plate (127), and a connecting bolt (140) that fixes the protective body (120) to a fixture (200) by passing through the through hole (124) to be fastened to the fixture (200) configured to be implanted in an alveolar bone of a subject, and when the protective body (120) is fixed to the fixture (200) by the connecting bolt (140) in a process of implant placement surgery, the cover part (129) is configured to cover and protect a bone graft material (320) implanted around the fixture (200).

2. The screw of claim 1, wherein the horizontal plate (127) is formed in a fan shape.

3. The screw of claim 1, wherein the body (122) has a fixture connection part (126) formed on a lower part thereof, with the fixture connection part (126) having a thickness decreasing gradually downward, and the fixture connection part (126) is configured to be inserted into and fixed to the fixture (200).

4. The screw of claim 1, wherein the connecting bolt (140) has a sealing head (148) formed on a top thereof, and the sealing head (148) is configured to seal a gum that is pierced during implant placement surgery at a time of tooth extraction from the subject.
